(19) United States
(12) Patent Application Publication
(10) Pub. No.: US 2025/0245259 A1
(43) Pub. Date: Jul. 31, 2025

(54) SYSTEMS AND METHODS FOR TEXT DATA PROCESSING AND CHUNK DISTRIBUTION

(71) Applicant: LLAMALAB, INC., New York, NY (US)

(72) Inventor: Shere SAIDON, New York, NY (US)

(73) Assignee: LLAMALAB, INC., New York, NY (US)

(21) Appl. No.: 19/035,367

(22) Filed: Jan. 23, 2025

Related U.S. Application Data
(60) Provisional application No. 63/625,505, filed on Jan. 26, 2024.

(51) Int. Cl. G06F 16/353; G16H 50/70

(52) U.S. Cl. CPC G06F 16/353; G16H 50/70

(57) ABSTRACT

Disclosed herein are systems, methods, and media for processing and distributing text data from a dataset. The disclosed embodiments include receiving raw data and converting the raw data into a set of text chunks. The disclosed embodiments include determining a set of classifications for the raw data. The disclosed embodiments include. augmenting a text chunk in the set of text chunks with metadata. The disclosed embodiments include generating a windowed chunk by appending context to the augmented chunk. The disclosed embodiments include embedding the windowed chunk and the extracted retrieval metadata into a chunk embedding. The disclosed embodiments include distributing the chunk embedding by determining a data store in the set of data stores corresponding to the assigned classification and assigning the chunk embedding to the determined data store.

### CROSS-REFERENCE TO RELATED APPLICATIONS

This application claims priority to and the benefit of U.S. Provisional Patent Application No. 63/625,505, filed on Jan. 26, 2024. The disclosure of the above-referenced application is expressly incorporated herein in its entirety.

### FIELD OF DISCLOSURE

The disclosed embodiments generally relate to systems, devices,

methods, and computer readable media for processing and distributing text data from a dataset.

### BACKGROUND

Traditional or conventional data analysis platforms may be used for generating responses to dataset queries, such as a request to retrieve information from a dataset. However, conventional systems may be inefficient and/or inaccurate in ingesting, analyzing, and processing datasets including large amounts of data in various formats. For example, databases may store text data in different file formats, and conventional systems may not be able to efficiently comprehend and extract the relevant text data, including extracting relevant themes and commonalities in data, to generate insights from the data.

Further, conventional systems may not be capable of generating data stores customized to provide insights into complex questions or training tasks. For example, medical records may involve large amounts of unlabeled data that may be used to analyze patient information. However, conventional systems may process and retrieve inaccurate data or generate fake or spurious data (hallucinations) in response to a query, which may result in ill-informed and potentially harmful healthcare decisions.

### SUMMARY

Some disclosed embodiments include methods for processing and distribution text data from a dataset. Some disclosed embodiments involve receiving raw data and converting the raw data into a set of text chunks. Some disclosed embodiments involve determining a set of classifications for the raw data. In some embodiments, the set of classifications may correspond to a set of data stores. Some disclosed embodiments involve augmenting a text chunk in the set of text chunks with metadata. Some disclosed embodiments involve extracting retrieval metadata from the text chunk. Some disclosed embodiments involve determining and assigning, with a machine learning model configured to classify text information, a classification in the set of classifications for the text chunk. Some disclosed embodiments may involve sequencing the text chunk.

Some disclosed embodiments involve generating a windowed chunk by appending context to the augmented chunk. Some disclosed embodiments involve embedding the windowed chunk and the extracted retrieval metadata into a chunk embedding. Some disclosed embodiments involve distributing the chunk embedding. Some disclosed embodiments involve determining a data store in the set of data stores corresponding to the assigned classification. Some disclosed embodiments involve assigning the chunk embedding to the determined data store.

Some disclosed embodiments involve training the machine learning model with ground truth data. Some disclosed embodiments involve ordering the augmented text chunk. Some disclosed embodiments involve a general data store, and based on a determination that the text chunk is not assigned to a classification in the set of classifications, assigning the text chunk to the general data store.

In some embodiments, the determined data store comprises a repository. In some embodiments, the determined data store comprises a second machine learning model. In some embodiments, the raw data includes medical record data.

Other systems, methods, and computer-readable media are also discussed herein. Disclosed embodiments may include any of the above aspects alone or in combination with one or more aspects, whether implemented as a method, by at least one processor, and/or stored as executable instructions on non-transitory computer readable media.

### BRIEF DESCRIPTION OF THE DRAWINGS

The accompanying drawings, which are incorporated in and constitute a part of this specification, illustrate several embodiments and, together with the description, serve to explain the disclosed principles. In the drawings:

FIG. 1 illustrates a block diagram of processing inputs, consistent with embodiments of the present disclosure.

FIG. 2 illustrates a block diagram of input data processing and extraction, consistent with embodiments of the present disclosure.

FIG. 3 illustrates a block diagram of processing and distributing input data, consistent with embodiments of the present disclosure.

FIG. 4 illustrates a block diagram of chunk augmentation, consistent with embodiments of the present disclosure.

FIG. 5 illustrates a block diagram of data decomposition and query response synthesis, consistent with embodiments of the present disclosure.

FIG. 6 illustrates a method for processing and distributing text data, consistent with embodiments of the present disclosure.

FIG. 7 is a block diagram illustrating an exemplary operating environment for implementing various aspects of this disclosure, consistent with embodiments of the present disclosure.

FIG. 8 is a block diagram illustrating an exemplary machine learning platform for implementing various aspects of this disclosure, consistent with embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. In the following detailed description, numerous specific details are set forth in order to provide a thorough understanding of the disclosed example embodiments. However, it will be understood by those skilled in the art that the principles of the exemplary embodiments may be practiced without every specific detail. Well-known methods, procedures, and components have not been described in detail so as not to obscure the principles of the example embodiments. Unless explicitly stated, the example methods and processes described herein are neither constrained to a particular order or sequence nor constrained to a particular system configuration. Additionally, some of the described embodiments or elements thereof can occur or be performed (e.g., executed) simultaneously, at the same point in time, or concurrently. Reference will now be made in detail to the disclosed embodiments, examples of which are illustrated in the accompanying drawings.

It is to be understood that both the foregoing general description and the following detailed description are exemplary and explanatory only and are not restrictive of this disclosure. The accompanying drawings, which are incorporated in and constitute a part of this specification, illustrate several exemplary embodiments and together with the description, serve to outline principles of the exemplary embodiments.

This disclosure may be described in the general context of customized hardware capable of executing customized preloaded instructions such as, e.g., computer-executable instructions for performing program modules. Program modules may include one or more of routines, programs, objects, variables, commands, scripts, functions, applications, components, data structures, and so forth, which may perform particular tasks or implement particular abstract data types. The disclosed embodiments may also be practiced in distributed computing environments where tasks are performed by remote processing devices that are linked through a communications network. In a distributed computing environment, program modules may be located in local and/or remote computer storage media including memory storage devices.

Disclosed embodiments may provide improvements to the functioning of a computer process, database, or a machine learning system by improving data ingestion, data analysis, and retrieval of information from any data, including data stored in datasets and databases. To generate analytics, insights, and responses to queries, datasets with large amounts of data may be used. Some disclosed embodiments may involve queries, questions, prompts, or retrieval of information from data, as well as generating responses to questions based on data. In some examples, data may include information that has been written, typed, or spoken, including Optical Character Recognition performed on handwritten documents, scanned documents, and transcriptions of speech. Data may include data stored in databases which may be structured (e.g., SQL) or unstructured, as well as in file formats such as CSV, PDFs, or JavaScript Object Notation (JSON). In another example, data may include data stored in the cloud. In some embodiments, analyzing data may involve generating responses to complex queries. For example, the disclosed embodiments may involve decomposing queries and data, as well as synthesizing queries and data, to generate an answer for a query. It will be recognized that analyzing data as described herein may include processing large amounts of data which may be stored in a variety of formats, which is a computationally complex task.

Embodiments of the present disclosure may be utilized to generate insights and make data-driven decisions with quick and accurate analysis, thereby enabling effective use of data for optimized operations. The disclosed embodiments may include processing data related to any field, including information stored or represented as text (e.g., language written, stored, or typed). In some examples, datasets may include information specialized (e.g., specific or particular) to a certain field. In some embodiments, datasets may include data related to legal information. For example, legal information may include case law, regulatory data, legislative data, and data related to a legal proceeding (e.g., documents such as contracts, briefs, motions, emails, and letters). In some embodiments, datasets may include data related to medical (e.g., health) related information, including patient record data. For example, medical record data may include electronic health records. In some examples, medical record data may refer to data of a single individual (e.g., a single patient's medical history). In another example, medical record data may refer to data of multiple individuals, such as data which has been aggregated across multiple patients or data from patients sharing a similar characteristic (e.g., a shared condition). The disclosed embodiments may provide improvements to healthcare decisions based on data as well as generating custom algorithms that can revolutionize the medical record analytics field. It will be appreciated that any examples directed to medical record data discussed herein are merely exemplary, and the embodiments described herein are not limited to medical data.

Some disclosed embodiments may involve processing and distributing data from a dataset. In some embodiments, data processing may involve any preparation, transformation, or organization of data (e.g., information), such as data manipulation or adjustment. In some examples, processing may involve preparing data for presentation to a module, such as a machine learning model. Processing may also involve splitting apart data. Data distribution may refer to the allocation of information, including delivery, assignment, or dispersal of data. In some examples, data distribution may involve the transfer of information (e.g., between modules). Some disclosed embodiments may involve receiving data. Receiving data may include at least one of retrieving, requesting, receiving, acquiring, or obtaining information.

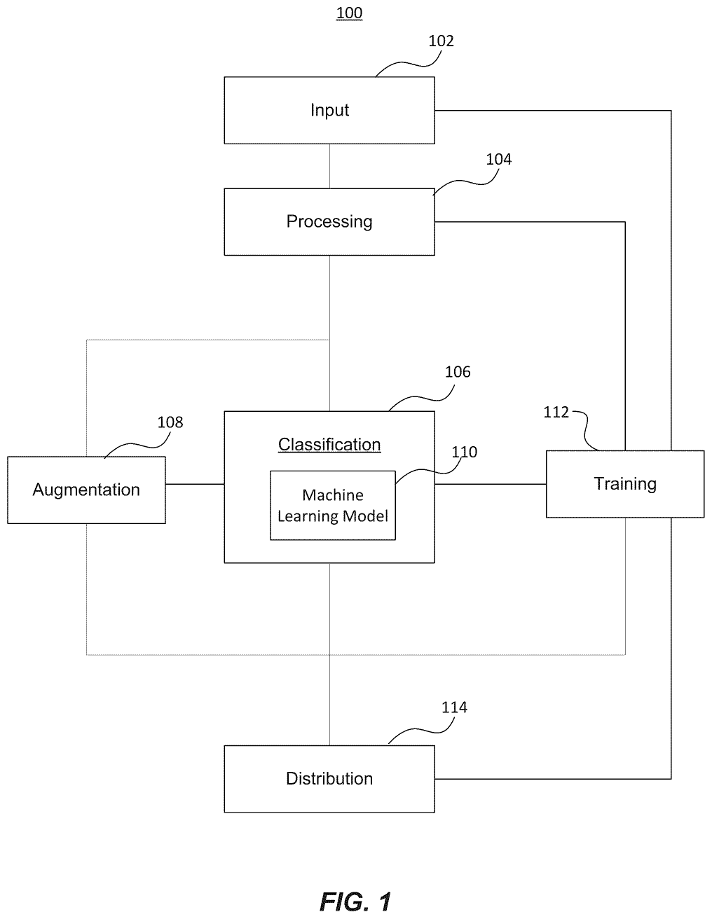

FIG. 1 illustrates a block diagram 100 of processing inputs, consistent with embodiments of the present disclosure. Input 102 may refer to any data in a dataset, such as data stored in a corpus, database, repository, or the like. Input 102 may include data in one or more documents stored in a database, including digital files as well as hard copy (e.g., printed or handwritten on paper) documents. Input 102 may include data stored in spreadsheets, scanned paper documents, document management systems, the internet, the cloud, and public and/or private repositories. Input 102 may refer to raw data, such as the native or original form of data stored in a database. In some examples, input 102 may be text data, such as any data represented in a textual format. Text data may include natural language, such as printed, typed, or written numbers, words, phrases, paragraphs, or the like. For example, input 102 may be a handwritten paragraph describing a person's occupation in a scanned document stored in a database.

Block diagram 100 may include a step of processing 104. Processing, as described herein, may include conversions and/or transformations between data formats. Processing may involve data ingestion, such as the collection and preparation of data for analysis. Processing 104 may involve preparing input data for presentation to a computer-implemented component, such as converting raw data to a format that a computer can process. For example, raw data in input 102 may be transformed into structured data that may be readable by a machine. In the example of handwritten data, the handwritten data may be scanned (e.g., to a Portable Document Format (PDF)) and optical character recognition (OCR) may be performed on the PDF to convert the handwritten data into machine-encoded text. Processing 104 may also involve decomposing data (e.g., breaking up data), such as creating subsets of data. Data may be decomposed into smaller components in order to improve processing (e.g., improve computational speed and conserve memory). For example, decomposed data may refer to breaking a document into multiple pages, a paragraph into multiple sentences, or a sentence into multiple words.

In some embodiments, input data may be classified. For example, block diagram 100 may include classification 106. Classification may refer to the determination of categories, groupings, or designations for data. Classification may also involve assigning or identifying data to one or more classes (e.g., categories). For example, classification may involve receiving data, determining categories of information based on the data, and assigning the data to the category. Classification may involve assigning a label (e.g., a category) to decomposed data. For example, sentences in a paragraph may be assigned the classification of “sentence.” In some embodiments, classification 106 may involve a machine learning model 110. For example, machine learning model 110 may be any model configured to classify data, including binary (or multi-class) classifiers, neural networks, transformers, regression models, support vector machines, decision trees, random forests, nearest-neighbor, as well as clustering models, as non-limiting examples. Machine learning model 110 may classify input data 102 and/or processed data. In an example, machine learning model 110 may determine classifications, such as determining categories and the number of categories, for data. Additionally, or alternatively, machine learning model 110 may assign data or decomposed data to a classification. In another example, machine learning model 110 may be a generative machine learning model. In some examples, machine learning model 110 may represent a request to a machine learning model, such as an application programming interface (API) call to a machine learning model.

In some embodiments, block diagram 100 may include augmentation 108. Augmentation may refer to enhancements performed on data, such as any adjustment or modifications to improve data. Augmentation may involve modifying data with additional data (e.g., data from a same data source or a different data source) in order to enhance the value or usefulness of data. In some examples, augmentation may be performed before or alongside classification (e.g., classification 106). In the example of a machine learning model performing classification, augmentation performed before classification may enhance the data being classified, thereby providing a more robust dataset to the machine learning model and enabling a more robust classification. Additionally, or alternatively, augmentation 108 may be performed after classification. For example, after classification, augmentation may involve enhancing data assigned to various categories. In some embodiments, augmentation may involve metadata augmentation.

Metadata may refer to data which may provide information about other data. Metadata may provide details about characteristics of data, such as the identification, structure, and/or context of data. For example, metadata may include descriptive, administrative, reference metadata, or the like. Additional examples of metadata may include data properties, history, origin, and versions. In some embodiments, augmentation 108 may involve metadata augmentation. Metadata augmentation may refer to augmenting data with metadata, such as enhancing data with additional information. For example, metadata augmentation may involve tagging or associating descriptive keywords or contextual information with data. In one example, metadata may provide additional context and information about data, enabling improved analysis and categorization (e.g., by a machine learning model). In another example, metadata may provide additional context and enable improved retrieval from data categories (e.g., after classification).

In some embodiments, block diagram 100 may involve distribution 114. Distribution, as described herein, may refer to allocation and/or transmission of data. For example, distribution may involve allocating data based on classifications assigned to data. In some examples, data may be distributed to a machine learning model for further analysis or processing, distributed to a database for storage, or presented to a device (e.g., a user interface). It will be appreciated that data distribution may enable improved analysis of data as well as improvements to responses generated for answering queries (e.g., questions) to datasets and any insights generated from datasets.

In some embodiments, block diagram 100 may involve training 112. Training a machine learning model may refer to configuring or updating the machine learning model, such as by adjusting, learning, or optimizing parameters of the model (e.g., determining weights for layers in the model). For example, training may refer to updating the classification 106, such as updating the types or number of categories. Training may also involve training the classification 106 and/or machine learning model 110. In some examples, training may be based on one or more of input 102, processing 104, augmentation 108, and/or distribution 114. For example, the training of the model may be updated based on inputs in input 102.

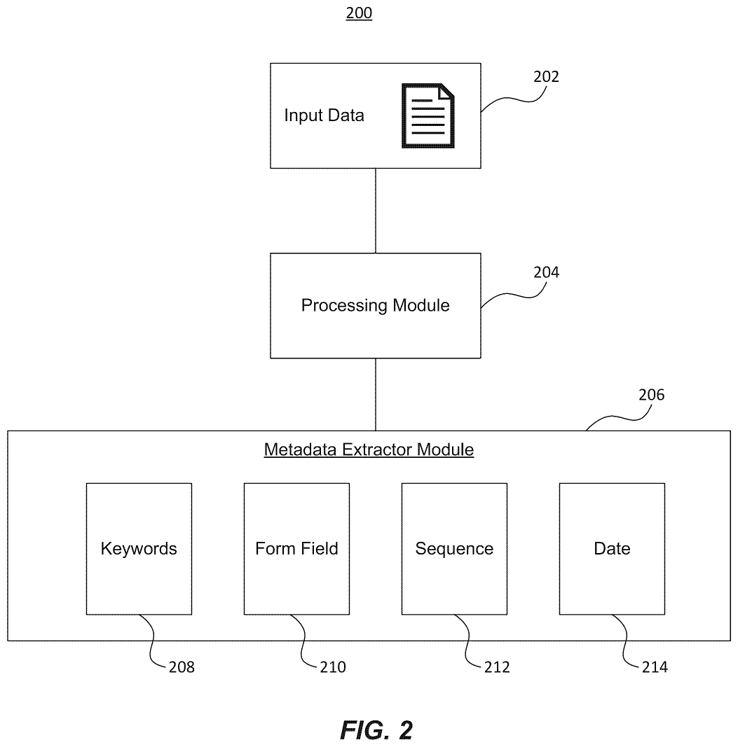

FIG. 2 illustrates a block diagram 200 of processing and extracting data, consistent with embodiments of the present disclosure. As described herein, input data 202 may be data stored in a database. In some examples, input data 202 may include raw data. For example, input data 202 may include raw data stored as text data in PDFs. Processing Module 204 may refer to a module for preparing input data 202 for analysis, as described herein. Processing module 204 may convert input data 202 to another format. For example, processing module 204 may convert raw data into a format that can be used by a machine learning model. In another example, processing 204 may involve converting input data 202, including raw data, into smaller data components. In some embodiments, processing module 204 may convert information into a format configured for analysis by a computer. For example, processing module 204 may include a OCR module to convert scanned or handwritten information, or a transcription module to convert text to speech.

In some embodiments, data may be converted to a standardized (e.g., normalized) format. It will be recognized that data and/or metadata may exist in various forms, which may be inefficient for processing. Some disclosed embodiments may involve standardizing data and/or metadata to a standard format. For example, data such as dates may be inputted or received in different date formats. In another example, keywords, abbreviations, acronyms, and synonyms for text data may be converted to a standard format, such as by standardizing all synonyms for a specific word to a standard word (e.g., doctor, practitioner, and clinician may be standardized to physician). Data may also be normalized by extracting keywords, key points, or summaries extrapolated from input text. In some embodiments, a machine learning model may convert information to standardized formats. For example, a machine learning model in processing module 204 may be configured to standardize data and/or metadata. In another example, machine learning models configured for natural language processing (NLP) tasks as described herein may be used for standardization. As such, it will be appreciated that regardless of the input format, data and/or metadata may be converted to a standardized format.

It will be recognized that input data 202 may include metadata. The metadata may describe various features and characteristics of input data 202, such as file properties, structures (e.g., formats), keywords, and dates. Input data, including both raw data and converted data, may involve metadata. Some disclosed embodiments may involve extracting metadata. Extracting may refer to retrieving information, including obtaining and/or selecting data. For example, extracting metadata may include identifying and collecting metadata from input data 202. Metadata may also be extracted from processed data, such as data which has been converted (e.g., by processing module 204) and/or split into sub-data (e.g., by a classifier). Some disclosed embodiments may involve a metadata extractor module 206, which may be any module configured to extract metadata. In some embodiments, metadata extractor module 206 may involve one or more sub-modules which may be customized to process and extract specific types of metadata. Metadata extractor module 206 may include sub-modules involving machine learning models, such as any model configured to automatically extract data. In some embodiments, metadata extractor module 206 may include a classifier model to classify data (e.g., machine learning model 110 as referenced in FIG. 1) and/or metadata. Additionally, or alternatively, metadata extractor module 206 may involve one or more software modules, such as software libraries.

In some embodiments, metadata extractor module 206 may include a keyword extractor 208. Keyword extractor 208 may be a sub-module for extracting keywords (e.g., characters, acronyms, words, or phrases) that may be relevant. In some examples, keyword extractor 208 may be a model trained to identify keywords. In another example, keyword extractor 208 may extract identified keywords, such as keywords that have been identified as relevant and compiled in a database. Keyword extractor 208 may include programs and modules to extract metadata, such as any natural language module. For example, keyword extractor 208 may include regular expression matching operations (e.g., regex patterns), Natural Language Toolkit (NLTK), or SpaCy. In the example of medical data, keyword extractor 208 may identify and extract medical keywords (e.g., “test result,” “glucose,” “emergency,” and “surgeon” as non-limiting examples).

In some embodiments, metadata extractor module 206 may include a form field extractor 210. Form field extractor 210 may extract form fields, which may be designated spaces or areas for collecting information. For example, a form field may be a checkbox on a PDF or a drop-down menu on a user interface containing information inputted by a user. Form field extractor 210 may use OCR to extract metadata. For example, form fields may contain metadata inputted by a patient and/or by a medical professional (e.g., form field extractor 210 may identify a field of “allergies” in a form upon detection of a checkbox list for a patient's allergies).

In some embodiments, metadata extractor module 206 may include a sequence extractor module 212. Sequence extractor module 212 may assist in extracting metadata related to the sequence, order, or position of data. The sequence of data may include how words are ordered next to each other in a sentence or the spatial positioning between paragraphs in a document. Sequence extractor module 212 may use OCR, for example, to identify the page number corresponding to data.

In some embodiments, metadata extractor module 206 may include a date extractor 214. Date extractor 214 may extract dates or other information based in time. For example, date extractor 214 may use regex or date parsing methods to retrieve dates corresponding to a patient's history of medical procedures, or in another example, to retrieve the date a document stored in input data 202 was created.

It will be recognized that the examples of sub-modules of metadata extractor module 206 are not limited to the aforementioned examples. Metadata extractor module 206 may be trained (e.g., updated) to improve sub-modules as well as to include additional sub-modules that may be needed based on the input data 202. It will be appreciated that sub-modules may improve the accuracy of identifying and retrieving metadata and improve the speed of metadata processing, thereby improving machine learning model training, output, and accuracy for models trained with the metadata.

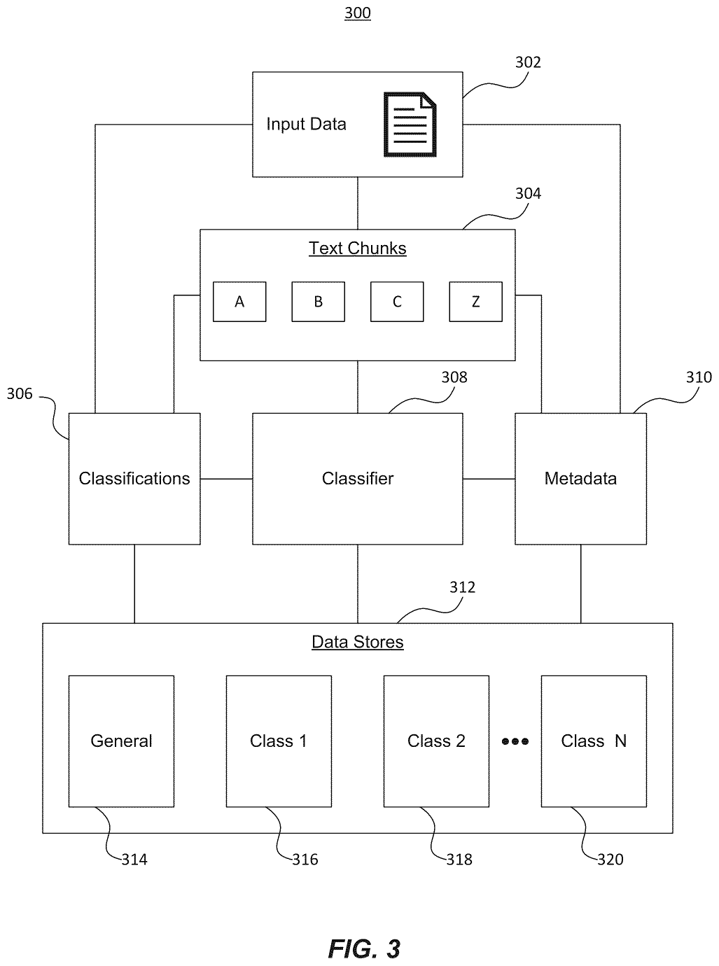

FIG. 3 illustrates a block diagram 300 of processing and distributing input data, consistent with embodiments of the present disclosure. As described herein, input data 302 may include raw data, which may be broken down into smaller data. Some disclosed embodiments involve converting raw data into a set of text chunks 304. Block diagram 300 may illustrate examples also demonstrated in FIG. 1 (e.g., conversion of data into smaller units of data (tokenization), may be an example of processing 104). A text chunk may refer to a subset of text data, such as a unit of natural language that has been extracted or broken from a larger unit of language. A text chunk may be a segment or piece of text that may be handled as an individual unit. For example, a text chunk may refer to a part of a word, an entire word, a group of words, a phrase, a sentence, or a collection of sentences. Raw data may be converted into text chunks 304 by various methods of chunking including tools and/or rules to determine factors such as chunk length. Text chunks 304 may include any number of text chunks. In some embodiments, natural language processing (NLP) tools may be used to convert input data into chunks. For example, NLP tools may include modules such as NLTK. Conversion into chunks may involve grouping or dividing inputs based on patterns such as semantics (e.g., meanings and relations between words) as well as syntactic chunking (e.g., based on grammar or sentence structure). In some embodiments, machine learning models may be used for chunking, such as any machine learning model configured for NLP tasks, including Long Short-Term Memory networks or transformer-based models. In some embodiments, conversion into chunks may involve rule-based chunking, which may be determined by specific rules or criteria. The chunks may be determined by certain attributes or values based on the rules. In an example, OCR tools may involve placing markers to identify stop and/or start points for chunks (e.g., information in a table or within a page may be considered as a chunk). In another example, each row in a CSV file may be determined as a chunk. In an additional example, a stop indicator, such as punctuation, a gap during speech, or a specific keyword, may be used to determine chunks. Data may also be converted to chunks based on a predetermined chunk length (e.g., a number of characters) or number of chunks. For example, data may be broken into a total of 256 or 512 chunks (corresponding to 256 bytes tokens). It will be appreciated that chunking, including for large amounts of texts across various documents with differing formats, may be a computationally complex task. For example, the human mind may not be equipped to perform analysis of large amounts of chunks corresponding to large amounts of text data. Further, it will be appreciated that dividing input data into text chunks may enable easier analysis for a machine learning model. For example, text chunking may improve the management of system memory use and processing speed, as smaller text chunks may use less model bandwidth than larger amounts of text.

Some disclosed embodiments may involve determining a set of classifications for data. A set of classifications may refer to categories determined for data. Classifications 306 may include the classifications (e.g., names, types, and number of categories) for input data 302. In some examples, classifications 306 may include a set of classifications for raw data in input data 302. In some embodiments, classifications in classifications 306 may be determined by a classifier 308, which may be any machine learning model configured to classify data. In some examples, classifier 308 may represent classification 106 and/or machine learning model 110, as referenced in FIG. 1. For example, classifier 308 may receive input data 302, and classifier 308 may determine classifications 306 based on input data 302. In an example, classifier 308 may analyze the raw data to determine the classifications and the number of classifications, and classifications 306 may be updated as the classifier analyzes the input data. Additionally, or alternatively, classifications 306 may be determined based on attributes of input data 302. For example, the set of classifications in classifications 306 may be determined based on keywords or attributes (e.g., including attributes that have been predetermined). In an example corresponding to medical record data, determined classifications may include types of medical record data, such as diagnoses, providers, physician notes, allergies, and test results. In some embodiments, classifications 306 may also include a general classification. For example, the general classification may be a category for classifying data that may not fit (e.g., assigned to) to a specific classification.

Some disclosed embodiments may involve classifying a text chunk. Classifying a text chunk, such as a chunk in the set of chunks 304, may involve assigning a text chunk to a category, such as a classification in the set of classifications 306. In some embodiments, classifier 308 may classify a text chunk. Classifier 308 may be any machine learning model configured to classify text information, as described herein. For example, classifier 308 may be a large language model, which may be a model (e.g., transformer model) configured to understand, interpret, generate, and/or respond to language. The large language model may be seeded with a categorization prompt. In some embodiments, training a machine learning model may involve ground truth data. Classifier 308 may be trained with ground truth documentation and classifications for the ground truth. For example, the ground truth may include medical record data of providers for different patients, and the classifications may be the type of provider (e.g., specialist, surgeon, primary care). Training machine learning models may also involve training supervised learning models (e.g., regression models, neural networks, transformers), unsupervised learning models (e.g., clustering) and/or active learning models. In some examples, classifier 308 may be a fine-tuned model, such as a model which has been trained with a specialized or specific dataset (e.g., specific to legal documents or medical records). In some embodiments, classifying may involve determining and assigning a classification. For example, classifier 308 may determine a class (or multiple classes) for a text chunk in text chunks 304. Assigning the classification may involve associating the text chunk with the determined class. For example, based on the decision of classifier 308, a text chunk may be assigned a label corresponding to a classification in the set of classifications 306. In some examples, determining and/or assigning may involve patterns learned by the machine learning model, statistics (e.g., probability or confidence scores), and/or threshold determinations.

In some embodiments, the set of classifications 306 may correspond to a set of data stores 312. A data store may refer to any component or module capable of receiving data and/or inputs. Data stores, in some examples, may be configured to contain data. In some embodiments, a data store may include a repository, which may be any method of storage, such as a database, corpus, drive, or cloud. In some embodiments, a data store may include a machine learning model. For example, a data store may be a machine learning model configured to further process and/or analyze data. In some examples, machine learning models included in a data store may be the same or different from classifier 308. For example, machine learning models included in a data store may be a second machine learning model. The set of data stores 312 may include one or more data stores corresponding to classifications 306. For example, data stores 312 may include a first data store 316 corresponding to a first classification, a second data store 318 corresponding to a second classification, and any additional N data store 320 corresponding to any number of determined classifications. As the classifications in classifications 306 may be adjusted (e.g., reducing categories, adding categories, or modifying individual categories), data stores 312 may adjust accordingly. The set of data stores 312 may include a general data store 314, which may receive data that is not assigned to first data store 316, second data store 318, or any other N data store 320, as an example (e.g., general data store 314 may receive data determined to have a classification of “other”).

Some disclosed embodiments may involve distributing data, as described herein. For example, data may be distributed to a data store, such as a data store of data stores 312. A data store may receive data according to the classification corresponding to the data store. As described herein, a data store in set of data stores 312 may correspond to a classification in classifications 306. As such, a data store may receive data corresponding to an assigned classification for the data. For example, a text chunk in the set of text chunks 304 may be assigned (e.g., by classifier 308) a classification of first classification, and the text chunk may be distributed to the data store 316 corresponding to first classification.

Text chunks in the set of text chunks 304 may be augmented with metadata 310. Augmenting, as described herein, may involve enhancing, such as enhancing a text chunk in the set of text chunks 304. In some embodiments, augmenting a text chunk with metadata may involve extracting retrieval metadata from the text chunk. Retrieval metadata may refer to any metadata, as described herein, which can assist in retrieval and/or distribution of data. For example, retrieval metadata may include information regarding the identification, description, searching, indexing, organization, and formatting of data. In some embodiments, retrieval metadata may be extracted from text chunks 304 (e.g., from data that has been processed). Additionally, or alternatively, retrieval metadata may be extracted from input data 302, including raw data. Retrieval metadata may include any metadata extracted, such as with metadata extractor module 206, as referenced in FIG. 2. The retrieval metadata may be used to augment a text chunk by being associated with a text chunk. For example, retrieval metadata may be tagged or appended to a text chunk. In another example, retrieval metadata may be mapped to a data structure or space with the text chunk, or the retrieval metadata may be embedded within the text chunk. It will be appreciated that metadata extraction may involve the analysis and processing of various formats of metadata in varying volumes (including large volumes), which may be a computationally complex task. Further, it will be appreciated that metadata extraction may be a digitally-implemented task which the human mind is not equipped to perform.

In some embodiments, a text chunk in the set of text chunks 304 may be sequenced before distribution to a data store. Sequencing may involve determining the spatial or relative position of data, such as the positioning of words or sentences, as described herein. For example, sequence extractor module 212 (as referenced in FIG. 2) may determine the sequencing of text chunks by determining relative positions between chunks. In another example, sequence extractor module 212 may determine the position of a text chunk in relation to a page of a document in the input data. In some embodiments, sequencing may involve using retrieval metadata. The retrieval metadata may enhance the sequential information for a chunk, such as by providing additional information stored as metadata that may assist in sequencing. In an example, the retrieval metadata may include the page of where the chunk originated, which may help in sequencing the text chunk in relation to other text chunks from the same page. It will be appreciated that sequencing chunks may provide improved retrieval of unique sets of text chunks. For example, sequencing a text chunk may reduce classifier redundancy, as sequencing the chunk and providing retrieval metadata to the chunk may indicate that the text chunk has already been analyzed by the classifier (e.g., classifier 308). By reducing unnecessary classifier redundancy, the bandwidth of the model may be lowered, thereby enabling efficiency and memory improvements (e.g., reducing unnecessary storage and processing of duplicate information). Further, sequencing with retrieval metadata may reduce unnecessary duplicate information that may skew the model, thereby improving the accuracy of the classifier model.

Some disclosed embodiments may involve ordering a text chunk. Ordering a text chunk may refer to organizing text chunks based on referenced relationships between chunks. Referenced relationships may include relationships or associations between different chunks, and the referenced relationships may provide an enhanced understanding of context, links, and hierarchy in the data. For example, a referenced relationship may provide information on different words that may refer to the same object or how different sections in a document relate to each other. In the example of medical record data, a referenced relationship may associate the hierarchical relationship between “testing results” and “complete blood count” (e.g., “complete blood count” is within the hierarchy of “testing results”). Ordering may involve sorting text chunks based on the referenced relationships. For example, ordering may involve sorting the text chunk based on text chunks determined to have a referenced relationship (e.g., the ordering may place the text chunk proximal to another text chunk sharing a referenced relationship). As such, ordering may involve organizing text chunks such that text chunks with referenced relationships may be positioned (e.g., in a virtual representation space or in a database) closer together than text chunks without referenced relationships. It will be appreciated that ordering may assist in providing efficiency and improvements to retrieval and distribution of text chunks.

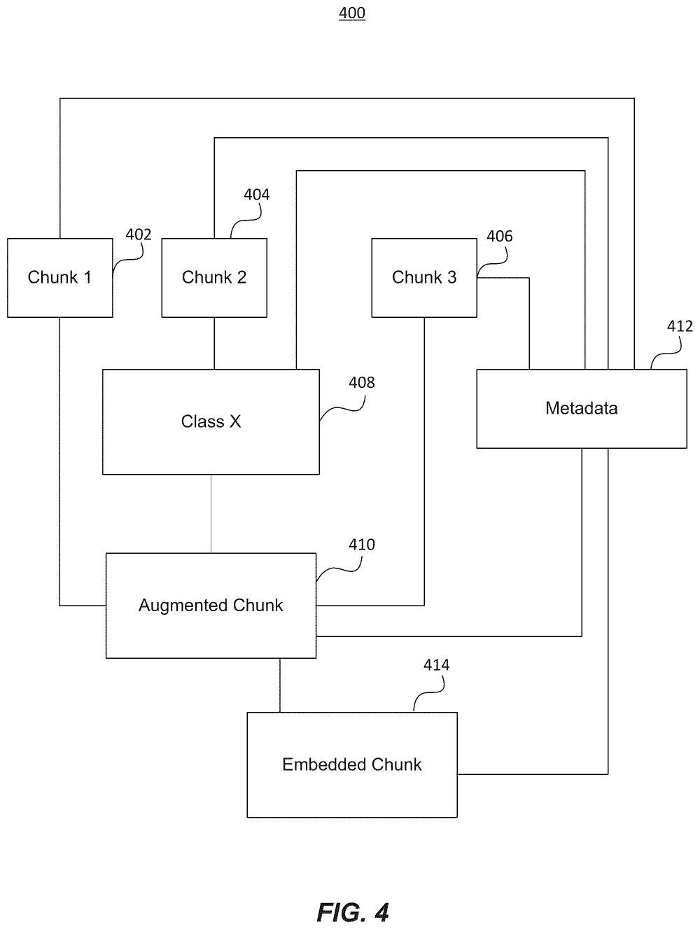

FIG. 4 illustrates a block diagram 400 of chunk augmentation, consistent with embodiments of the present disclosure. In some embodiments, block diagram 400 may illustrate ordering of text chunks, such as first chunk 402, second chunk 404, and third chunk 406. For example, chunk 404 may be assigned a classification 408 (e.g., by a classifier such as classifier 308) and augmented with other chunks in the input data and the referential relationships of the chunks. Based on the referenced relationships, ordering may involve adding chunk 402 (e.g., a previous chunk in the ordering) and chunk 406 (e.g., the next chunk in the ordering) to chunk 404, thereby generating an augmented chunk 410. The text chunks and/or the referenced relationships may be added to chunk 404. As such, a retrieved chunk (e.g., chunk 404) may be augmented with surrounding chunks. Additionally, or alternatively, block diagram 400 may illustrate generating a windowed chunk. In some embodiments, the windowed chunk may be generated by creating a text window around the text chunk. A text window may refer to fixed (e.g., predetermined) size or number of data points. For example, by surrounding an individual text chunk with a given number of data points, a text window may be generated. In some embodiments, the windowed chunk may be generated by appending context to a text chunk. Context may refer to additional data which may be proximal to a data point, such as any data surrounding the data point. For example, given a sentence in a paragraph, context may refer to other sentences in the paragraph surrounding the sentence (e.g., before and/or after). Context may refer to data itself, as well as metadata. In some embodiments, context may be generated through tokenization. Modules and/or machine learning models as described herein may be used to tokenize text around a given data point, such as by using a module to process and divide a paragraph into individual sentences. For example, chunk 404 may be a text chunk in a paragraph, and NLTK may be used to tokenize the paragraph and generate tokens of context, such as words or sentences from the paragraph. The context may be appended to chunk 404, thereby generating the windowed chunk, which may be augmented chunk 410. Providing additional context may assist in improving chunk retrieval. For example, augmented chunk 410 may be a windowed chunk appended with any number of data points (e.g., 20 words or 20 sentences before and after the text chunk). In some embodiments, appending may involve concatenation, such as the concatenation of strings (e.g., adding strings representing the context to a string representing the text chunk). In another example, appending may involve converting context and/or text chunks to a vector format (e.g., using an NLP library) and adding the context to the text chunk in the vectorized representation.

Some disclosed embodiments involve embedding a text chunk, and additionally or alternatively, embedding metadata. An embedding may refer to any alternative or converted representation of data, such as a mathematical representation of input data. Embeddings may include transformations of data into formats which computerized systems, such as machine learning models, may be able to understand and process. In some examples, embeddings may include a vector representation of data, such as a data structure (e.g. vector, array, tensor) where data points may be represented in a numerical format. Embeddings (e.g., encodings) may include latent space representations, such as any compressed feature space and/or space where data with similar features are mapped closer together in the space. For example, two words having similar meanings or originating from the same page in a document may be mapped closer together in an embedding space. As such, embeddings may provide dimensionality reduction of data by storing data in a computationally-efficient manner, thereby conserving memory and improving computational bandwidth for machine learning models. In some embodiments, a machine learning model may generate an embedding (e.g., machine learning model 110). For example, a module including Word2Vec or a transformer model may generate an embedding, or an application programming interface (API) may be configured to generate embeddings. In another example, embeddings may be generated by an embedding layer in a neural network model. Referring to FIG. 4, an embedded chunk may be generated by embedding augmented chunk 410 into embedded chunk 414. For example, a windowed chunk and/or an ordered chunk may be embedded into embedded chunk 414. In some embodiments, metadata 412 may be embedded into an embedding. For example, the generated embedded chunk 414 may involve embedding augmented chunk 410 along with metadata 412. Metadata 412 may include metadata from text chunks, such as text chunk 402, second text chunk 404, or third text chunk 406. For example, metadata 412 may include retrieval metadata extracted from any text chunk. Additionally, or alternatively, metadata 412 may include metadata from input data, as described herein.

In some embodiments, an embedded chunk may be distributed to a data store. For example, embedded chunk 414 may be distributed to a data store in the set of data stores 312, as referenced in FIG. 3. The embedded chunk may be distributed to a datastore corresponding to a classification in the set of classifications 306, as described herein. For example, a text chunk in the set of text chunks classified by classifier 308 as belonging to a “test results” class in classifications 306 may be converted into a text chunk embedding including metadata 310, and the embedded text chunk may be distributed to a “test results” data store in the set of data stores 312. It will be appreciated that embedding metadata along with a text chunk may improve retrieval of the text chunk. For example, the set of text chunks 304 may include a large amount of text chunks, and metadata 310 may include large quantities of retrieval metadata from the text chunks and/or input data 302. Embedding the text chunks may improve the distribution and processing of such large quantities of data. Further, it will be appreciated that as the data stores in the set of data stores 312 may each contain text chunks corresponding to a specific class, the data stores may be specialized according to the corresponding class. As such, the data stores may be used to train and/or generate specialized machine learning models for each classification. For example, custom algorithms may be trained for various medical datasets. Thus, embedding the text chunk with metadata may provide enhanced machine learning models and data sets for training machine learning models, which may thereby result in improved training of the machine learning models as well as improved accuracy for output generation. For example, a “test results” data store containing embedded text chunks and metadata may provide an improved data set to train a “test results” machine learning model. In another example, a text chunk may be assigned a classification of “other” or “general” (e.g., not classified as a specific category in the set of classifications 306). Such a text chunk may be transformed to a “general” embedded text chunk and distributed to a general data store 314, which may reduce the amount of unclassified data distributed to the specialized data stores, thereby reducing data contamination and enhancing the specificity of the data stores. As such, it will be appreciated that aspects of the disclosed embodiments improve model training and accuracy. Further, classifying text chunks and distributing the text chunks to data stores may reduce hallucination (e.g., generating false, fake, or nonsensical information) for responses generated based on information retrieved from data as described herein, as well as reducing hallucination for machine learning models trained with the data in the data stores.

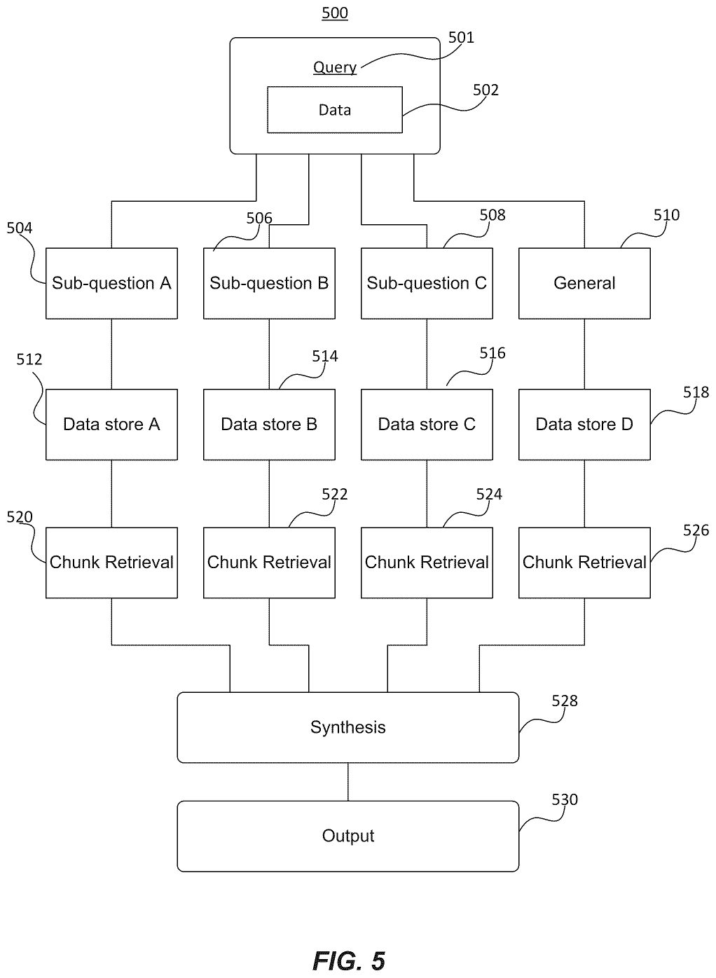

As described herein, disclosed embodiments may involve improvements to retrieving information from datasets in order to generate responses to queries. FIG. 5 illustrates a block diagram of data decomposition and query response synthesis, consistent with embodiments of the present disclosure. For example, a query 501 to a dataset 502 may be any request for information, including a question, prompt, problem, or the like. In some examples, query 501 may be received via an interface (e.g., a user interface) or received as an input (e.g., through a keyboard or speech-text interface). In some embodiments, a query 501 may be decomposed into sub-queries (e.g., sub-questions). Decomposition may involve breaking query 501 into simpler and/or smaller sub-queries. In an example where dataset 502 includes medical record data, a query 501 to dataset 502 may be complex, such as “evaluate the patient's recent weight loss.” The query 501 may be decomposed into multiple sub-questions, such as a first sub- question 504, a second sub-question 506, and a third sub-question 508, as well as a general sub-question 510. In some embodiments, query 501 may be decomposed into sub-questions by a machine learning model, including a large language model. The machine learning model may generate sub-questions according to various factors or components of the questions which should be answered to provide an accurate output. In some embodiments, query 501 may be generated into sub-questions based on various rules and prompts. In the example, a large language model may decompose the query by generating sub-questions, such as determining that the first sub-question 504 may be “what are the patient's recent weight measurements and blood test results,” second sub-question 506 may be “what is the patient's medication history,” the third sub-question may be “what is the patient's family weight loss history,” and general sub-question 510 may include “evaluate previous notes for patient diet changes.” In some examples, a sub-query may be further decomposed, such as first sub-question 504 decomposed into ‘recent weight measurements” and “blood test results.” It will be appreciated that by decomposing the query 501 into sub-questions, accurate data may be retrieved for each sub-question, thereby assisting in retrieval information relevant to the query.

In some embodiments, the sub-questions may be assigned to a data-store for data retrieval. For example, first sub-question 504 may be assigned to first data store 512, second sub-question 506 to data store 514, third sub-question 508 to third data store 516, and general sub-question 510 to a general data store 518. The data stores may correspond to data stores in the set of data stores 312, as referenced in FIG. 3. For example, first data store 512 may refer to first data store 316, second data store 515 may refer to data store 318, third data store 516 may refer to data store 320, and general data store 518 may refer to general data store 314. As such, the sub-questions may be assigned to a data store specialized for information associated with the data store (and thereby the classification of the data store). Thus, it will be appreciated that the sub-questions may be routed to the appropriate data store including data specialized to answer the sub-question, enabling efficient and accurate processing, distribution, and analysis for large amounts of data. For example, text chunks in each data store may be retrieved, such as first chunks 520 retrieved from first data store 512, second chunks 522 retrieved from second data store 514, third chunks 524 retrieved from third data store 516, and general chunks 526 retrieved from general data store 518.

As described herein, the retrieved data chunks may be embedded chunks. For example, for a given sub-question, any number of embedded chunks may be retrieved, such as one, five, ten, or more chunks. In some examples, chunks may be retrieved by algorithms and/or machine learning models including search vector machines, cosine similarity, logistic regression, and maximal marginal relevance. It will be appreciated that as the chunks may be classified and augmented with metadata, the information generated from the retrieved chunks may have improved accuracy. In some embodiments, the chunks retrieved for each sub-question may by synthesized. Synthesis 528 may involve generating a response (e.g., answer) to one or more sub-questions based on information retrieved from the text chunks. For example, a large language model may use the retrieved text chunks from the data stores to generate a response to the sub-questions. In some embodiments, output 530 may be the generated response to the query 501. The output 530 may be generated by synthesizing and/or summarizing the responses to the sub-questions in order to generate the response. In the example of medical record data, the responses to the sub questions may be “the patient's test results are normal,” “the patient has started taking a medication with a side effect of altering metabolism,” “the patient has no family history of weight loss issues,” and “the patient has reported no appetite changes.” As such, a large language model may generate the output 530 of “the weight loss may be caused by medication side effects.” In another example, output 530 may be a generated summary of a patient's medical history.

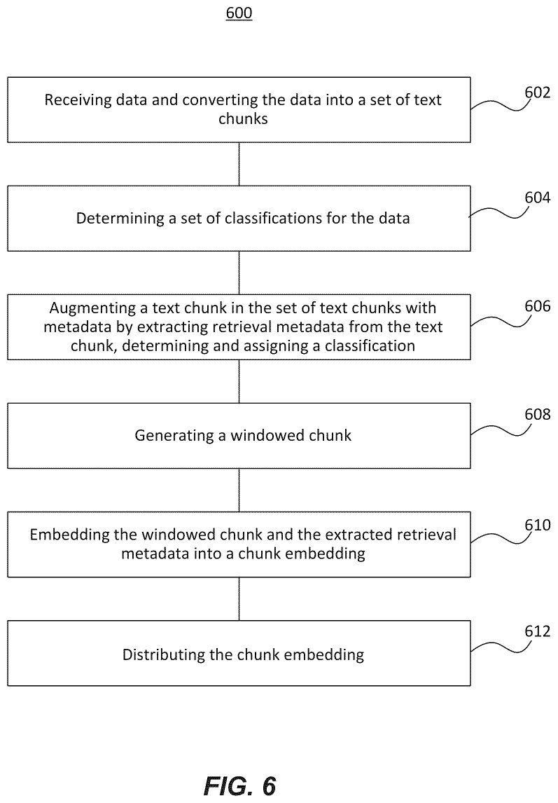

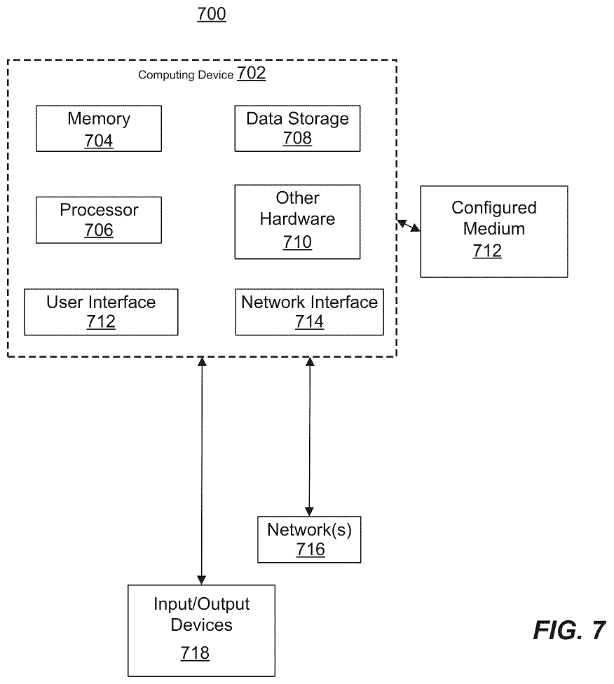

FIG. 6 illustrates a flow chart of a method 600 for processing and distributing text data, consistent with embodiments of the present disclosure. For convenience of description, method 600 may be described herein as being performed by a computer, such as computing device 702 as referenced in FIG. 7. However, the disclosed embodiments are not so limited. In some embodiments, method 600 may be performed by one or more processors, microprocessors, or computing systems. For example, method 600 may be performed by processor 706. Furthermore, the computer(s) used for training machine learning model described herein may differ or be separate from the computer(s) used to obtain the training data, the computer(s) used to generate the training dataset, or the computer(s) which may use the machine learning model for inference.

In some embodiments, method 600 may include a step 602 of receiving data and converting the data into a set of text chunks. Data may include any data in data sets, such as datasets which may include large amounts of information. In some examples, step 602 may involve receiving raw data and converting the raw data into a set of text chunks. In an example, raw data may include medical record data stored in a database.

In some embodiments, method 600 may include a step 604 of determining a set of classifications for the data. In some embodiments, a machine learning models may determine the classifications based on the raw data and/or the text chunks. In some examples, the set of classifications may correspond to a set of data stores. The data stores may include machine learning models and/or repositories. In the example of medical record data, the classifications may correspond to types or categories of medical record data, such as medications, providers, history, and test results.

In some embodiments, method 600 may include a step 606 of augmenting a text chunk in the set of text chunks with metadata. In some examples, the metadata may be extracted from a text chunk or multiple text chunks in the set of text chunks. For example, retrieval metadata may be extracted from a text chunk. In some embodiments, metadata may also be extracted from the input data. In some embodiments, step 606 may involve determining and assigning a classification in the set of classifications for the text chunk. For example, a classifier may determine categories for the text chunks and assign a text chunk to the category. In the example of medical record data, metadata may include descriptions of the format of files in the dataset, such as PDFs. In some embodiments, augmenting the text chunk may involve sequencing the text chunk. In some embodiments, step 606 may include ordering the text chunk. For example, ordering the text chunk may involve organizing text chunks based on relationships between different text chunks.

In some embodiments, method 600 may involve a step 608 of generating a windowed chunk. A windowed chunk may be a chunk combined with additional data. For example, generating a windowed chunk may involve appending context to the augmented chunk, such as appending a text chunk with sentences surrounding it.

In some embodiments, method 600 may include a step 610 of embedding the windowed chunk. Embedding the windowed chunk may refer to transforming the chunk into a vector representation. For example, a machine learning model may embed a chunk into an embedding. In some embodiments, a windowed chunk as well as metadata, such as retrieval metadata, may be embedded into a chunk embedding.

In some embodiments, method 600 may include a step 612 of distributing the chunk embedding. Step 612 may involve determining a data store in the set of data stores corresponding to the assigned classification. Step 612 may involve assigning the chunk embedding to the determined data store. For example, for a chunk assigned to the classification of “test results,” the embedded chunk may be distributed to the data store corresponding to “test results.”

An exemplary operating environment for implementing various aspects of this disclosure is illustrated in FIG. 7. As illustrated in FIG. 7, an exemplary operating environment 700 may include a computing device 702 (e.g., a general-purpose computing device) in the form of a computer. Components of the computing device 702 may include, but are not limited to, various hardware components, such as one or more processors 706, data storage 708, a system memory 704, other hardware 710, and a system bus (not shown) that couples (e.g., communicably couples, physically couples, and/or electrically couples) various system components such that the components may transmit data to and from one another. The system bus may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

With further reference to FIG. 7, an operating environment 700 for an exemplary embodiment includes at least one computing device 702. The computing device 702 may be a uniprocessor or multiprocessor computing device. An operating environment 700 may include one or more computing devices (e.g., multiple computing devices 702) in a given computer system, which may be clustered, part of a local area network (LAN), part of a wide area network (WAN), client-server networked, peer-to-peer networked within a cloud, or otherwise communicably linked. A computer system may include an individual machine or a group of cooperating machines. A given computing device 702 may be configured for end-users, e.g., with applications, for administrators, as a server, as a distributed processing node, as a special-purpose processing device, or otherwise configured to train machine learning models and/or use machine learning models.

One or more users may interact with the computer system comprising one or more computing devices 702 by using a display, keyboard, mouse, microphone, touchpad, camera, sensor (e.g., touch sensor) and other input/output devices 718, via typed text, touch, voice, movement, computer vision, gestures, and/or other forms of input/output. An input/output device 718 may be removable (e.g., a connectable mouse or keyboard) or may be an integral part of the computing device 702 (e.g., a touchscreen, a built-in microphone). A user interface 712 may support interaction between an embodiment and one or more users. A user interface 712 may include one or more of a command line interface, a graphical user interface (GUI), natural user interface (NUI), voice command interface, and/or other user interface (UI) presentations, which may be presented as distinct options or may be integrated. A user may enter commands and information through a user interface or other input devices such as a tablet, electronic digitizer, a microphone, keyboard, and/or pointing device, commonly referred to as mouse, trackball or touch pad. Other input devices may include a joystick, game pad, satellite dish, scanner, or the like. Additionally, voice inputs, gesture inputs using hands or fingers, or other NUI may also be used with the appropriate input devices, such as a microphone, camera, tablet, touch pad, glove, or other sensor. These and other input devices are often connected to the processing units through a user input interface that is coupled to the system bus, but may be connected by other interface and bus structures, such as a parallel port, game port or a universal serial bus (USB). A monitor or other type of display device is also connected to the system bus via an interface, such as a video interface. The monitor may also be integrated with a touch-screen panel or the like. Note that the monitor and/or touch screen panel can be physically coupled to a housing in which the computing device is incorporated, such as in a tablet-type personal computer. In addition, computers such as the computing device may also include other peripheral output devices such as speakers and printer, which may be connected through an output peripheral interface or the like.

One or more application programming interface (API) calls may be made between input/output devices 718 and computing device 702, based on input received from at user interface 712 and/or from network(s) 716. As used throughout, “based on” may refer to being established or founded upon a use of, changed by, influenced by, caused by, or otherwise derived from. In some embodiments, an API call may be configured for a particular API, and may be interpreted and/or translated to an API call configured for a different API. As used herein, an API may refer to a defined (e.g., according to an API specification) interface or connection between computers or between computer programs.

System administrators, network administrators, software developers, engineers, and end-users are each a particular type of user. Automated agents, scripts, playback software, and the like acting on behalf of one or more people may also constitute a user. Storage devices and/or networking devices may be considered peripheral equipment in some embodiments and part of a system comprising one or more computing devices 702 in other embodiments, depending on their detachability from the processor(s) 706. Other computerized devices and/or systems not shown in FIG. 7 may interact in technological ways with computing device 702 or with another system using one or more connections to a network 716 via a network interface 714, which may include network interface equipment, such as a physical network interface controller (NIC) or a virtual network interface (VIF).

Computing device 702 includes at least one logical processor 706. The at least one logical processor 706 may include circuitry and transistors configured to execute instructions from memory (e.g., memory 704). For example, the at least one logical processor 706 may include one or more central processing units (CPUs), arithmetic logic units (ALUs), Floating Point Units (FPUs), and/or Graphics Processing Units (GPUs). The computing device 702, like other suitable devices, also includes one or more computer-readable storage media, which may include, but are not limited to, memory 704 and data storage 708. In some embodiments, memory 704 and data storage 708 may be part a single memory component. The one or more computer-readable storage media may be of different physical types. The media may be volatile memory, non-volatile memory, fixed in place media, removable media, magnetic media, optical media, solid-state media, and/or of other types of physical durable storage media (as opposed to merely a propagated signal). In particular, a configured medium 720 such as a portable (i.e., external) hard drive, compact disc (CD), Digital Versatile Disc (DVD), memory stick, or other removable non-volatile memory medium may become functionally a technological part of the computer system when inserted or otherwise installed with respect to one or more computing devices 702, making its content accessible for interaction with and use by processor(s) 706. The removable configured medium 720 is an example of a computer-readable storage medium. Some other examples of computer-readable storage media include built-in random access memory (RAM), read-only memory (ROM), hard disks, and other memory storage devices which are not readily removable by users (e.g., memory 704).

The configured medium 720 may be configured with instructions (e.g., binary instructions) that are executable by a processor 706; “executable” is used in a broad sense herein to include machine code, interpretable code, bytecode, compiled code, and/or any other code that is configured to run on a machine, including a physical machine or a virtualized computing instance (e.g., a virtual machine or a container). The configured medium 720 may also be configured with data which is created by, modified by, referenced by, and/or otherwise used for technical effect by execution of the instructions. The instructions and the data may configure the memory or other storage medium in which they reside; such that when that memory or other computer-readable storage medium is a functional part of a given computing device, the instructions and data may also configure that computing device.

Although an embodiment may be described as being implemented as software instructions executed by one or more processors in a computing device (e.g., general-purpose computer, server, or cluster), such description is not meant to exhaust all possible embodiments. One of skill will understand that the same or similar functionality can also often be implemented, in whole or in part, directly in hardware logic, to provide the same or similar technical effects. Alternatively, or in addition to software implementation, the technical functionality described herein can be performed, at least in part, by one or more hardware logic components. For example, and without excluding other implementations, an embodiment may include other hardware logic components 710 such as Field-Programmable Gate Arrays (FPGAs), Application-Specific Integrated Circuits (ASICs), Application-Specific Standard Products (ASSPs), System-on-a-Chip components (SOCs), Complex Programmable Logic Devices (CPLDs), and similar components. Components of an embodiment may be grouped into interacting functional modules based on their inputs, outputs, and/or their technical effects, for example.

In addition to processor(s) 706, memory 704, data storage 708, and screens/displays, an operating environment may also include other hardware 710, such as batteries, buses, power supplies, wired and wireless network interface cards, for instance. The nouns “screen” and “display” are used interchangeably herein. A display may include one or more touch screens, screens responsive to input from a pen or tablet, or screens which operate solely for output. In some embodiment, other input/output devices 718 such as human user input/output devices (screen, keyboard, mouse, tablet, microphone, speaker, motion sensor, etc.) will be present in operable communication with one or more processors 706 and memory.

In some embodiments, the system includes multiple computing devices 702 connected by network(s) 716. Networking interface equipment can provide access to network(s) 716, using components (which may be part of a network interface 714) such as a packet-switched network interface card, a wireless transceiver, or a telephone network interface, for example, which may be present in a given computer system. However, an embodiment may also communicate technical data and/or technical instructions through direct memory access, removable non-volatile media, or other information storage-retrieval and/or transmission approaches.

The computing device 702 may operate in a networked or cloud-computing environment using logical connections to one or more remote devices (e.g., using network(s) 716), such as a remote computer (e.g., another computing device 702). The remote computer may include one or more of a personal computer, a server, a router, a network PC, or a peer device or other common network node, and may include any or all of the elements described above relative to the computer. The logical connections may include one or more LANs, WANs, and/or the Internet.

When used in a networked or cloud-computing environment, computing device 702 may be connected to a public or private network through a network interface or adapter. In some embodiments, a modem or other communication connection device may be used for establishing communications over the network. The modem, which may be internal or external, may be connected to the system bus via a network interface or other appropriate mechanism. A wireless networking component such as one comprising an interface and antenna may be coupled through a suitable device such as an access point or peer computer to a network. In a networked environment, program modules depicted relative to the computer, or portions thereof, may be stored in the remote memory storage device. It may be appreciated that the network connections shown are exemplary and other means of establishing a communications link between the computers may be used.

Computing device 702 typically may include any of a variety of computer-readable media. Computer-readable media may be any available media that can be accessed by the computer and includes both volatile and nonvolatile media, and removable and non-removable media, but excludes propagated signals. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, electrically erasable programmable read-only memory (EEPROM), flash memory or other memory technology, CD-ROM, DVD or other optical disk storage, magnetic cassettes, magnetic tape, magnetic disk storage or other magnetic storage devices, or any other medium which can be used to store the desired information (e.g., program modules, data for a machine learning model, and/or a machine learning model itself) and which can be accessed by the computer. Communication media may embody computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of the any of the above may also be included within the scope of computer-readable media. Computer-readable media may be embodied as a computer program product, such as software (e.g., including program modules) stored on non-transitory computer-readable storage media.

The data storage 708 or system memory includes computer storage media in the form of volatile and/or nonvolatile memory such as ROM and RAM. A basic input/output system (BIOS), containing the basic routines that help to transfer information between elements within computer, such as during start-up, may be stored in ROM. RAM may contain data and/or program modules that are immediately accessible to and/or presently being operated on by processing unit. By way of example, and not limitation, data storage holds an operating system, application programs, and other program modules and program data.

Data storage 708 may also include other removable/non-removable, volatile/nonvolatile computer storage media. By way of example only, data storage may be a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

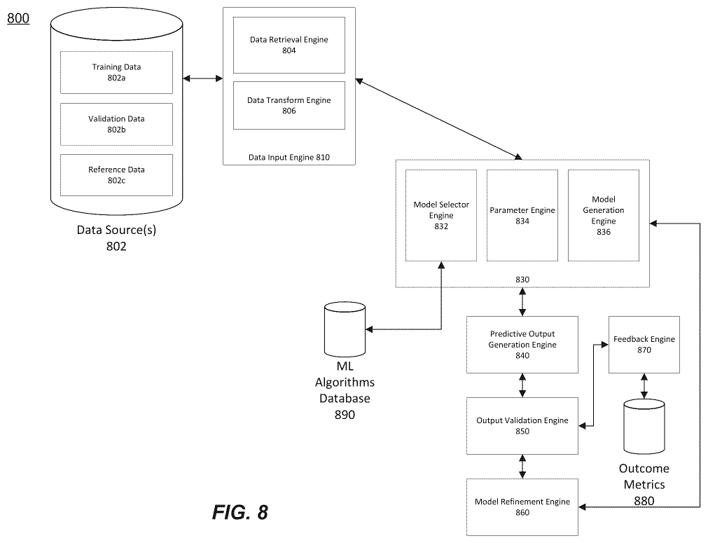

Exemplary disclosed embodiments include systems, methods, and computer readable media for the generation of text and/or code embeddings. For example, in some embodiments, and as illustrated in FIG. 7, an operating environment 700 may include at least one computing device 702, the at least one computing device 702 including at least one processor 706, at least one memory 704, at least one data storage 708, and/or any other component discussed above with respect to FIG. 8.

FIG. 8 is a block diagram illustrating an exemplary machine learning platform for implementing various aspects of this disclosure, according to some embodiments of the present disclosure.

System 800 may include data input engine 810 that can further include data retrieval engine 804 and data transform engine 806. Data retrieval engine 804 may be configured to access, access, interpret, request, or receive data, which may be adjusted, reformatted, or changed (e.g., to be interpretable by other engine, such as data input engine 810). For example, data retrieval engine 804 may request data from a remote source using an API. Data Input engine 810 may be configured to access, interpret, request, format, re-format, or receive input data from data source(s) 802. For example, data input engine 810 may be configured to use data transform engine 806 to execute a re-configuration or other change to data, such as a data dimension reduction. Data source(s) 802 may exist at one or more memories 704 and/or data storages 708. In some embodiments, data source(s) 802 may be associated with a single entity (e.g., organization) or with multiple entities. Data source(s) 802 may include one or more of training data 802a (e.g., input data to feed a machine learning model as part of one or more training processes), validation data 802b (e.g., data against which at least one processor may compare model output with, such as to determine model output quality), and/or reference data 802c. For example, training data 802a, validation data 802b, and/or reference data 802c may include data domains, as described herein. In some embodiments, data input engine 810 can be implemented using at least one computing device (e.g., computing device 702). For example, data from data sources 802 can be obtained through one or more I/O devices and/or network interfaces. Further, the data may be stored (e.g., during execution of one or more operations) in a suitable storage or system memory. Data input engine 810 may also be configured to interact with data storage 708, which may be implemented on a computing device that stores data in storage or system memory. System 800 may also include machine learning (ML) modeling engine 830, which may be configured to execute one or more operations on a machine learning model (e.g., model training, model re-configuration, model validation, model testing), such as those described in the processes described herein. In an example, machine learning modeling engine 830 may include machine learning model 110, as referenced in FIG. 1, and/or classifier 308 as referenced in FIG. 3. For example, ML modeling engine 830 may execute an operation to train a machine learning model, such as adding, removing, or modifying a model parameter. Training of a machine learning model may be supervised, semi-supervised, or unsupervised. In some embodiments, training of a machine learning model may include multiple epochs, or passes of data (e.g., training data 802a) through a machine learning model process (e.g., a training process). In some embodiments, different epochs may have different degrees of supervision (e.g., supervised, semi-supervised, or unsupervised). Data into to a model to train the model may include input data (e.g., as described above) and/or data previously output from a model (e.g., forming recursive learning feedback). A model parameter may include one or more of a seed value, a model node, a model layer, an algorithm, a function, a model connection (e.g., between other model parameters or between models), a model constraint, or any other digital component influencing the output of a model. A model connection may include or represent a relationship between model parameters and/or models, which may be dependent or interdependent, hierarchical, and/or static or dynamic. The combination and configuration of the model parameters and relationships between model parameters discussed herein are cognitively infeasible for the human mind to maintain or use. Without limiting the disclosed embodiments in any way, a machine learning model may include millions, trillions, or even billions of model parameters. ML modeling engine 830 may include model selector engine 832 (e.g., configured to select a model from among a plurality of models, such as based on input data), parameter selector engine 834 (e.g., configured to add, remove, and/or change one or more parameters of a model), and/or model generation engine 836 (e.g., configured to generate one or more machine learning models, such as according to model input data, model output data, comparison data, and/or validation data). ML algorithms database 880 (or other data storage 708) may store one or more machine learning models, any of which may be fully trained, partially trained, or untrained. A machine learning model may be or include, without limitation, one or more of (e.g., such as in the case of a metamodel) a statistical model, an algorithm, a neural network (NN), a convolutional neural network (CNN), a generative neural network (GNN), a Word2Vec model, a bag of words model, a term frequency-inverse document frequency (tf-idf) model, a GPT (Generative Pre-trained Transformer) model (or other autoregressive model), a Proximal Policy Optimization (PPO) model, a nearest neighbor model (e.g., k nearest neighbor model), a linear regression model, a k-means clustering model, a Q-Learning model, a Temporal Difference (TD) model, a Deep Adversarial Network model, or any other type of model described further herein.

System 800 can further include predictive output generation engine 840, output validation engine 850 (e.g., configured to apply validation data to machine learning model output), feedback engine 870 (e.g., configured to apply feedback from a user and/or machine to a model), and model refinement engine 860 (e.g., configured to update or re-configure a model). In some embodiments, feedback engine 870 may receive input and/or transmit output (e.g., output from a trained, partially trained, or untrained model) to outcome metrics database 8 70. Outcome metrics database 8 70 may be configured to store output from one or more models, and may also be configured to associate output with one or more models. In some embodiments, outcome metrics database 8 70, or other device (e.g., model refinement engine 860 or feedback engine 870) may be configured to correlate output, detect trends in output data, and/or infer a change to input or model parameters to cause a particular model output or type of model output. In some embodiments, model refinement engine 860 may receive output from predictive output generation engine 840 or output validation engine 850. In some embodiments, model refinement engine 860 may transmit the received output to ML modelling engine 830 in one or more iterative cycles.

Any or each engine of system 800 may be a module (e.g., a program module), which may be a packaged functional hardware unit designed for use with other components or a part of a program that performs a particular function (e.g., of related functions). Any or each of these modules may be implemented using a computing device. In some embodiments, the functionality of system 800 may be split across multiple computing devices to allow for distributed processing of the data, which may improve output speed and reduce computational load on individual devices. In some embodiments, system 800 may use load-balancing to maintain stable resource load (e.g., processing load, memory load, or bandwidth load) across multiple computing devices and to reduce the risk of a computing device or connection becoming overloaded. In these or other embodiments, the different components may communicate over one or more I/O devices and/or network interfaces.

System 800 can be related to different domains or fields of use. Descriptions of embodiments related to specific domains, such as natural language processing or language modeling, is not intended to limit the disclosed embodiments to those specific domains, and embodiments consistent with the present disclosure can apply to any domain that utilizes predictive modeling based on available data.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Example embodiments are described above with reference to flowchart illustrations or block diagrams of methods, apparatus (systems) and computer program products. It will be understood that each block of the flowchart illustrations or block diagrams, and combinations of blocks in the flowchart illustrations or block diagrams, can be implemented by computer program product or instructions on a computer program product. These computer program instructions may be provided to a processor of a computer, or other programmable data processing apparatus to produce a machine, such that the instructions, which execute via the processor of the computer or other programmable data processing apparatus, create means for implementing the functions/acts specified in the flowchart or block diagram block or blocks.

These computer program instructions may also be stored in a computer-readable medium that can direct one or more hardware processors of a computer, other programmable data processing apparatus, or other devices to function in a particular manner, such that the instructions stored in the computer-readable medium form an article of manufacture including instructions that implement the function/act specified in the flowchart or block diagram block or blocks.

The computer program instructions may also be loaded onto a computer, other programmable data processing apparatus, or other devices to cause a series of operational steps to be performed (e.g., executed) on the computer, other programmable apparatus or other devices to produce a computer implemented process such that the instructions that execute on the computer or other programmable apparatus provide processes for implementing the functions/acts specified in the flowchart or block diagram block or blocks.

Any combination of one or more computer-readable medium(s) may be utilized. The computer-readable medium may be a non-transitory computer-readable storage medium. In the context of this document, a computer-readable storage medium may be any tangible medium that can contain or store a program for use by or in connection with an instruction execution system, apparatus, or device.

Program code embodied on a computer-readable medium may be transmitted using any appropriate medium, including but not limited to wireless, wireline, optical fiber cable, RF, IR, etc., or any suitable combination of the foregoing.

Computer program code for carrying out operations, for example, embodiments may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider).

The flowchart and block diagrams in the figures illustrate examples of the architecture, functionality, and operation of possible implementations of systems, methods, and computer program products according to various embodiments. In this regard, each block in the flowchart or block diagrams may represent a module, segment, or portion of code, which includes one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts, or combinations of special purpose hardware and computer instructions.

It is understood that the described embodiments are not mutually exclusive, and elements, components, materials, or steps described in connection with one example embodiment may be combined with, or eliminated from, other embodiments in suitable ways to accomplish desired design objectives.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A method for processing and distributing text data from a dataset, the method comprising:
receiving raw data and converting the raw data into a set of text chunks;
determining a set of classifications for the raw data, the set of classifications corresponding to a set of data stores;
augmenting a text chunk in the set of text chunks with metadata, the augmenting comprising:
extracting retrieval metadata from the text chunk;
determining and assigning, with a machine learning model configured to classify text information, a classification in the set of classifications for the text chunk; and
sequencing the text chunk;
generating a windowed chunk by appending context to the augmented chunk;
embedding the windowed chunk and the extracted retrieval metadata into a chunk embedding; and
distributing the chunk embedding by:
determining a data store in the set of data stores corresponding to the assigned classification; and
assigning the chunk embedding to the determined data store.

2. The method of claim 1, further comprising training the machine learning model with ground truth data.

3. The method of claim 1, further comprising ordering the augmented text chunk.

4. The method of claim 1, further comprising a general data store, and based on a determination that the text chunk is not assigned to a classification in the set of classifications, assigning the text chunk to the general data store.

5. The method of claim 1, wherein the determined data store comprises a repository.

6. The method of claim 1, wherein the determined data store comprises a second machine learning model.

7. The method of claim 1, wherein the raw data comprises medical record data.

8. A machine learning system comprising:
at least one memory storing instructions;
at least one processor configured to execute the instructions to perform operations for processing and distributing text data from a dataset, the operations comprising:
receiving raw data and converting the raw data into a set of text chunks;
determining a set of classifications for the raw data, the set of classifications corresponding to a set of data stores;
augmenting a text chunk in the set of text chunks with metadata, the augmenting comprising:
extracting retrieval metadata from the text chunk;
determining and assigning, with a machine learning model configured to classify text information, a classification in the set of classifications for the text chunk; and
sequencing the text chunk;
generating a windowed chunk by appending context to the augmented chunk;
embedding the windowed chunk and the extracted retrieval metadata into a chunk embedding; and
distributing the chunk embedding by:
determining a data store in the set of data stores corresponding to the assigned classification; and
assigning the chunk embedding to the determined data store.

9. The system of claim 8, further comprising training the machine learning model with ground truth data.

10. The system of claim 8, further comprising ordering the augmented text chunk.

11. The system of claim 8, further comprising a general data store, and based on a determination that the text chunk is not assigned to a classification in the set of classifications, assigning the text chunk to the general data store.

12. The system of claim 8, wherein the determined data store comprises a repository.

13. The system of claim 8, wherein the determined data store comprises a second machine learning model. 14 The system of claim 8, wherein the raw data comprises medical record data.

15. A non-transitory computer-readable medium including instructions that are executable by one or more processors to perform operations comprising:
receiving raw data and converting the raw data into a set of text chunks;
determining a set of classifications for the raw data, the set of classifications corresponding to a set of data stores;
augmenting a text chunk in the set of text chunks with metadata, the augmenting comprising:
extracting retrieval metadata from the text chunk;
determining and assigning, with a machine learning model configured to classify text information, a classification in the set of classifications for the text chunk; and
sequencing the text chunk;
generating a windowed chunk by appending context to the augmented chunk;
embedding the windowed chunk and the extracted retrieval metadata into a chunk embedding; and
distributing the chunk embedding by:
determining a data store in the set of data stores corresponding to the assigned classification; and
assigning the chunk embedding to the determined data store.

16. The non-transitory computer readable medium of claim 15, further comprising training the machine learning model with ground truth data.

17. The non-transitory computer readable medium of claim 15, further comprising ordering the augmented text chunk.

18. The non-transitory computer readable medium of claim 15, further comprising a general data store, and based on a determination that the text chunk is not assigned to a classification in the set of classifications, assigning the text chunk to the general data store.

19. The non-transitory computer readable medium of claim 15, wherein the determined data store comprises a repository.

20. The non-transitory computer readable medium of claim 15, wherein the determined data store comprises a second machine learning model.
